(19) United States
(12) Patent Application Publication
(10) Pub. No.: US 2013/0064768 A1
(43) Pub. Date: Mar. 14, 2013

(54) LIPOPHILIC CATIONIC PROBE FOR PET-IMAGING

(71) Applicants: David K. Menon, Cambridge (GB); Michael P. Murphy, Newmarket (GB); Franklin I. Aigbirhio, London (GB); Robin A. Smith, Dunedin (NZ)

(72) Inventors: David K. Menon, Cambridge (GB); Michael P. Murphy, Newmarket (GB); Franklin I. Aigbirhio, London (GB); Robin A. Smith, Dunedin (NZ)

(73) Assignees: Medical Research Council, Swidon (GB); University of Otago, Dunedin (NZ); Cambridge Enterprise Limited, Cambridge (GB)

(21) Appl. No.: 13/636,106

(22) PCT Filed: Mar. 24, 2011
(86) PCT No.: PCT/GB2011/000425

§ 371 Date: Nov. 27, 2012

Related U.S. Application Data
(60) Provisional application No. 61/320,221, filed on Apr. 1, 2010.

(30) Foreign Application Priority Data
Apr. 1, 2010 (GB) 1005624.0

(51) Int. Cl. A61K 51/04; C07F 9/54

(52) U.S. Cl. 424/1.77; 568/9

(57) ABSTRACT

The present invention provides an imaging probe which comprises a lipophilic cation, a hydrophobic moiety and a PET nucleus. The present invention also provides a precursor molecule for the production of such an imaging probe and methods for using the probe for analysing mitochondrial membrane potential in a subject.

### FIELD OF THE INVENTION

The present invention relates to imaging probes for use in techniques such as positron-emission tomography (PET) and single photon emission computed tomography (SPECT) for the visualisation of mitochondrial energisation in vivo.

### BACKGROUND TO THE INVENTION

Mitochondrial dysfunction contributes to a wide range of pathologies, including cancer, diabetes, heart failure, cardiovascular and liver diseases, AIDS, autoimmune disorders, degenerative diseases and the pathophysiology of aging.

Diseases to which mitochondrial dysfunction contribute are often associated with significant changes in mitochondrial membrane potential (ΔΨm). Alteration in mitochondrial membrane potential is an important characteristic in pathologies that either involve suppressed apoptosis, such as cancer; or enhanced apoptosis, such as AIDS and degenerative diseases. It is also associated with the many diseases caused directly by mitochondrial dysfunction such as DNA mutations and oxidative stress.

There is thus a need to monitor mitochondrial function in patients.

Positron-emission tomography (PET) is a widely used technique to image biological tissues and metabolism within patients. A short-lived positron-emitting nucleus, such as ¹⁸F, is incorporated into a probe molecule and injected into a patient. The probe then accumulates in certain tissues. The location of the probe may be visualised from the gamma ray emission using a PET scanner, and the local concentration of the probes deduced from tomography.

Lipophilic cations such as tetra- or tri-phenylphosphonium cations penetrate the plasma and mitochondrial membranes and selectively accumulate in mitochondria because of the negative membrane potential across the inner membrane.

The use of tracers based on simple TPP cations for PET has been suggested for imaging mitochondrial dysfunction (Cheng et al (2005) J. Label. Compd. Radiopharm. 48:131-137), tumour imaging (Madar et al (1999) J. Nucl. Med. 40:1180-1185; Wang et al (2007) 50:5057-5069) and imaging for diagnosis of coronary artery disease (Madar et al (2006) J. Nucl. Med. 47:1359-1366).

The present invention relates to improvements to the known mitochondria-targeted PET probes.

### DESCRIPTION OF THE FIGURES

FIG. 1—Structures of the TPP cations used in the Examples.

FIG. 2—Time course of uptake of [³H]MitoQ into mouse tissues following iv injection. Mice were injected with a bolus of 100 nmol [³H]MitoQ by iv tail vein injection. At the indicated times the mice were killed and the [³H]MitoQ content in the tissues were determined. Data are in nmol MitoQ/g wet weight tissue and are means ± range for two separate mice per time point. A, liver and kidney, B, heart, muscle, brain and white adipose tissue (fat). C and D, view of the first 1 hour after injection of MitoQ for liver and kidney (C) and for heart, muscle, brain and white adipose tissue (fat) (D) respectively.

FIG. 3—Time course of clearance of [³H]TPP compounds from the circulation following iv injection. Mice were injected with a bolus of 100 nmol [³H]TPP compound by iv tail vein injection. At the indicated times the mice were killed and the [³H]TPP content in the blood were determined. Data are in nmol TPP compound/ml blood and are means ± range for two separate mice per time point. A, [³H]MitoQ; B, [³H]DecylTPP and [³H]FluoroUndecylTPP; C, [³H]TPMP.

FIG. 4—Time'course of uptake of [³H]DecylTPP and [³H]FluoroUndecylTPP into tissues. Mice were injected with a bolus of 100 nmol of [³H]DecylIPP or [³H]FluoroUndecylTPP by injection into the tail vein. At the indicated times the mice were killed and the content of [³H]DecyLTPP or [³H]FluoroUndecylTPP in the tissues determined. Data are means ± range for two separate mice per time point. A, liver and kidney for [³H]DecylTPP, B, heart, muscle, brain and white adipose tissue (fat) for [³H]DecylTPP , C, all tissues for [³H]FluoroUndecylTPP.

FIG. 5—Time course for uptake of [³H]TPMP into tissues. Mice were injected with a bolus of 100 nmol of [³H]TPMP by injection into the tail vein. At indicated times the mice were killed and the content of [³H]TPMP in the tissues determined.

Data are means ± range for two separate mice per time point. A, liver and kidney, B, heart, muscle, brain and white adipose tissue (fat).

FIG. 6—Comparison of uptake of TPP compounds into tissues at different times. Mice were injected with an iv bolus of 100 nmol of [³H]MitoQ, [³H]DecylTPP, [³H]FluoroUndecylTPP or [³H]TPMP and at 15 min (A, B), 1 h (C, D) or 5 h (E, F) the mice were killed and the tissue content of [³H]TPP compounds determined. Data are means ± range for two separate mice per time point and in A, C & E are nmol TPP compound/g wet weight tissue and in B, D & F are in nmol TPP compound/ml blood.

FIG. 7—Mitochondrial uptake of TPP compounds in vivo. A, Mice were injected with a bolus of 100 nmol [³H]FluoroUndecylTPP by injection into the tail vein.

After 15 min the mice were injected ip with DNP (200 or 300 μg/kg) or saline carrier and after a further 15 min later they were killed and the content of [³H]FluoroUndecylTPP in the tissues determined. Data are means ± range for two mice per condition. B, IAM-TPP is shown being taken up in to mitochondria within a cell where it reacts with thiol proteins to form a thioether adduct that can then be detected by immunoblotting. C, Confocal image of IAM-TPP binding to mitochondria in cells. C2C12 cells were incubated with 1 μM IAM-TPP for 3 h±10 μM FCCP. The cells were then fixed and the location of the TPP moiety within the cells determined by labelling with antiserum against the TPP moiety, visualised by immunofluorescence confocal microscopy. Control experiments confirmed that the IAM-TPP binding colocalised with the mitochondria-specific dye Mitotracker Orange (data not shown). D, Mice were injected with a bolus of 500 nmol of IAM-TPP by injection into the tail vein. After 1 h the mice were killed and liver and heart mitochondria were prepared. The mitochondria (40 μg protein) were separated by SDS-PAGE and proteins that had been labelled with IAM-TPP were detected by immunoblotting using antiserum against the TPP moiety. Mitochondria from mice that had not been exposed to IAM-TPP were used as controls. The experiment was repeated on three separate mice with similar results.

FIG. 8—Synthesis of the mitochondria-targeted PET probe 18F-FluoroUndecylTPP

### SUMMARY OF ASPECTS OF THE INVENTION

The present inventors have surprisingly found that if the hydrophobicity of the imaging probe is increased, for example by incorporating a hydrophobic moiety, this greatly increases the extent of accumulation in mitochondria and increases clearance of the probe from circulation, leading to a greater tissue:circulation ratio.

Together these factors mean that the hydrophobic mitochondria-targeted imaging probes of the present invention are 20-100 fold more sensitive and have better tissue loading and contrast properties than currently used imaging probes for the visualisation of mitochondrial energisation in vivo.

Thus, in a first aspect, the present invention provides an imaging probe which comprises a lipophilic cation, a hydrophobic moiety and a PET nucleus.

The imaging probe may be for use, for example, in positron-emission tomography (PET) and/or single photon emission computed tomography (SPECT)

The lipophilic cation may be or comprise triphenylphosphonium (TPP).

The hydrophobic moiety may be or comprise an aliphatic chain, for example an aliphatic chain comprising at least 5 carbon atoms.

The hydrophobic moiety may comprise a linear alkane chain, for example a linear decyl or undecyl chain.

The PET nucleus may, for example, be ¹⁸F.

The hydrophobic moiety may act as a linker between the lipophilic cation and the PET nucleus.

In a second aspect, the present invention provides a method for analysing mitochondrial membrane potential in a subject which comprises the following steps:
(i) administration of an imaging probe according to the first aspect of the invention to the subject;
(ii) visualisation of the probe; and
(iii) deduction of the absolute or relative magnitude of the mitochondrial membrane potential.

Mitochondrial membrane potential may be analysed, for example, to visualise tumours, investigate mitochondrial damage, diagnose/monitor a pathology which involves a change in mitochondrial energisation in a subject, or to investigate the effect of a test compound on mitochondrial potential.

In a third aspect, the present invention provides an imaging probe according to the first aspect of the invention for use in
(i) a method for analysing mitochondrial membrane potential;
(ii) a method for visualising tumours in a subject;
(iii) a method for investigating mitochondrial damage in a subject;
(iv) a method for diagnosing and/or monitoring a pathology in a subject; or
(v) a method for investigating the effect of a test compound on mitochondrial potential.

In a fourth aspect, the present invention provides a precursor molecule comprising a lipophilic cation and a hydrophobic moiety which can be reacted with an anionic form of the PET nucleus to produce an imaging probe according to the first aspect of the invention.

The precursor molecule may comprise a mesylate group which reacts with the anionic form of the PET nucleus. For example, the precursor molecule may be a mesylated alkyl triphenylphosphonium compound, reactable with ¹⁸F⁻to form ¹⁸F⁻FluoroalkylTPP.

In a fifth aspect, the present invention provides a method for producing an imaging probe according to the first aspect of the invention, which comprises the step of reacting an anionic form of the PET nucleus with a precursor molecule according to the fourth aspect of the invention.

The present invention also provides method for producing and administering an imaging probe according to the first aspect of the invention to a subject which comprises the following steps:
(i) synthesis of an anionic form of a PET nucleus, such as ¹⁸F⁻
(ii) incorporation of the anionic form of the PET nucleus into a precursor molecule according to any of the fourth aspect of the invention to produce an imaging probe;
(ii) administration of the Imaging probe to the subject.

The present invention also provides:
(i) a method for increasing the uptake of an imaging probe which comprises a triphenylphosphonium (TPP) cation which comprises the step of increasing the hydrophobicity of the imaging probe; and
(ii) a method for increasing the tissue:circulation ratio of an imaging probe which comprises a triphenylphosphonium (TPP) cation, which method comprises the step of increasing the hydrophobicity of the Imaging probe.

Hydrophobicity of the imaging probe may, for example, be increased by incorporation of an alkyl chain having at least 5 carbon atoms.

### DETAILED DESCRIPTION

### Positron-Emission Tomography (PET)

Positron emission tomography (PET) is a nuclear medicine imaging technique which produces a three-dimensional image or picture of functional processes in the body. The system detects pairs of gamma rays emitted indirectly by a positron-emitting radionuclide (tracer), which is introduced into the body on a biologically active molecule. Images of tracer concentration in 3-dimensional space within the body are then reconstructed by computer analysis. In modern scanners, this reconstruction is often accomplished with the aid of a CT X-ray scan performed on the patient during the same session, in the same machine.

The glucose analog fluorine-18 (F-18) fluorodeoxyglucose (FDG) is a biologically active molecule for PET which is widely used in clinical oncology. This tracer is taken up by glucose-using cells and phosphorylated by hexokinase, the concentrations of tracer imaged then give tissue metabolic activity, in terms of regional glucose uptake.

For imaging mitochondria and/or changes in mitochondrial membrane potential, it is possible to use lipophilic cations which selectively accumulate in mitochondria due to the negative inner membrane potential (−120 to −170 mV). Such lipophilic cations include rhodamine-123 (Rh123) and tetraphenyphosphonium salts.

The term “positron-emission tomography (PET) probe” or “imaging probe” used herein means a molecule suitable for use in positron-emission tomography, SPECT or any other imaging technique, which can be administered to a patient, for example, by injection, and which accumulates in a tissue of interest. The location and local concentration of the probe can then be deduced using PET scanning and tomography, SPECT or another type of imaging technque.

A mitochondria-targeted imaging probe selectively accumulates in mitochondria, possibly due to the high mitochondrial membrane potential. The uptake of the probe may be ΔΨₘ-dependent so that the probe can give information on the energisation status of mitochondria.

The probe of the present invention may have a distribution profile in the body which is a function of mitochondrial integrity.

The probes of the present invention may be useful for imaging variations in mitochondrial surface potential (ΔΨₘ) imaging cells or tissues having dysfunctional mitochondria and imaging or monitoring diseases or conditions associated with dysfunctional mitochondria.

The imaging probe of the present invention comprises a lipophilic cation, a hydrophobic moiety and a PET nucleus.

### Single Photon Emission Computed Tomography (SPECT)

Single photon emission computed tomography (SPECT) is a nuclear medicine tomographic imaging technique using gamma rays. It is very similar to conventional nuclear medicine planar imaging using a gamma camera, but is able to provide true 3D information. This information is typically presented as cross-sectional slices through the patient, but can be freely reformatted or manipulated as required.

The basic technique requires injection of a gamma-emitting radioisotope (also called radionuclide) into the bloodsteam of the patient. This may involve the attachment of a marker radioisotope to a ligand which is of interest for its chemical binding properties to certain types of tissues. This marriage allows the combination of ligand and radioisotope (the radiopharmaceutical) to be carried and bound to a place of interest in the body, which then (due to the gamma-emission of the isotope) allows the ligand concentration to be seen by a gamma-camera.

### Lipophilic Cation

The lipophilic cation moiety of the probe of the present invention may be any cation which accumulates in mitochondria due to the high mitochondrial membrane potential. The cation may have a delocalised positive charge which promotes its ΔΨₘ-dependent accumulation into mitochondria and its passage through phospholipid bilayers.

Examples of such cations include Rhodamine-123 and phosphonium cations, triphenyl and tetraphenyl phosphonium derivatives, arsonium derivatives, quaternary amines with hydrophobic groups e.g. tetrabenzyl ammonium, and hydrophobic aromatic systems with delocalised positive charges akin to rhodamine.

Several labelled phosphonium cations, such as [¹¹C]methyltriphenylphosphonium, ²²3-[¹⁸F]Fluoropropyl and 4-[¹⁸F]Fluorobenzyltriarylphosphonium have been used as mitochondrial targeting agents.

The lipophilic cation may be triphenylphosphonium which when linked to the hydrophobic moiety (see below) produces a lipophilic alkyl triphenylphosphonium cation.

### Hydrophobic Moiety

The hydrophobic moiety increases the overall hydrophobicity of the cation when associated with it, for example by covalent linkage. The the octanol/PBS partition coefficient of the imaging probe including the hydrophobic moiety may be at least 50, 100, 250, 500, 750 or 1000. The coefficients for MethylTPP and Decyl TPP are 0.35 and 5000 respectively where the larger the number reflects the higher hydrophobicity. FluoroUndecyl has a value of 740.

The hydrophobic moiety may, for example be an aliphatic chain. The aliphatic chain may comprise at least 2 carbon atoms, for example between 5 to 20, 8 to 15, or 10 to 12 carbon atoms. The aliphatic chain may have 10 or 11 carbon atoms.

The hydrophobic moiety may comprise an alkyl chain, which may be a substantially or completely linear alkyl chain, or include some branching. The chain may comprise one or more hetero atoms (e.g O, S, N, P) internally and or at the terminus. The hydrophobic moiety may, in addition, contain unsaturated (alkenyl, alkynyl, aryl, heteroaryl) components and/or may comprise one or more aromatic insertions.

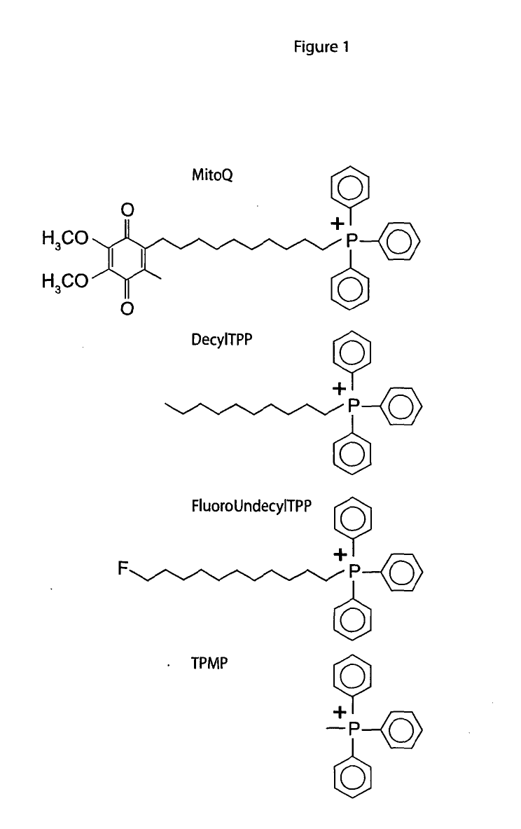

The hydrophobic moiety may be covalently linked to the lipophilic cation. Where the lipophilic cation is triphenylphosphonium (TPP) the hydrophobic moiety may be linked to the central phosphorus ion as shown in FIG. 1.

Without wishing to be bound by theory, the present inventors believe that the increased uptake associated with imaging probes comprising a hydrophobic moiety is due to more rapid permeation of the plasma membrane and the increased adsorption of the hydrophobic moiety to the matrix-facing surface of the mitochondrial inner membrane.

### Pet Nucleus

Non-radioactive elements and their counterparts that can be used in the probes of the present invention include: F-19 (F-18); C-12 (C-11); 1-127 (1-125, 1-124, 1-131 and 1-123); CI-36 (CI-32, CI-33, CI-34); Br-80 (Br-74, Br-75, Br-76, Br-77, Br-78); Re-185/187 (Re-186, Re-188); Y-89 (Y-90, Y-86); Lu-177 and Sm-153.

Alternatively the probes of the present invention may be labeled with one or more radio-isotopes, such as ¹¹C, ¹⁸F, ⁷⁶Br, ¹²³I, ¹²⁴I, ¹³¹I, ¹³N, or ¹⁵O.

Radionuclides used in PET scanning are typically isotopes with short half lives such as carbon-11 (˜20 min), nitrogen-13 (˜10 min), oxygen-15 (˜2 min), and fluorine-18 (˜110 min).

The PET nucleus may comprise ¹⁸F.

The term “PET nucleus” refers to a non-radioactive element or radionuclide which may be used in PET, SPECT or other imaging processes.

The PET nucleus may be attached, for example covalently linked to the lipophilic cation and/or the hydrophobic moiety. For example the hydrophobic moeity may act as a linker between the lipophilic cation and the PET nucleus.

The probe may be ¹⁸F-FluoroUndecylTPP.

### Method For Use

The present invention also provides a method for analysing mitochondrial membrane potential in a subject which comprises the following steps:
(i) administration of an imaging probe according to the present invention to the subject;
(ii) visualisation of the probe; and
(iii) deduction of the mitochondrial membrane potential.

The imaging apparatus used to detect and monitor the imaging agent include imaging technologies such as gamma camera, PET apparatus and SPECT apparatus.

Analysis of the mitochondrial membrane potential may be used in, for example, diagnosing and/or monitoring a pathology which involves a change in mitochondrial energisation in a subject.

Analysis of the mitochondrial membrane potential may be used in, for example, a method for visualising tumours or a method for investigating mitochondrial damage in a subject.

The probe may be administered by any suitable technique known in the art, such as direct injection. Injection may be intravenous (IV). Administration may be general or local to the site of interest, such as to a tumour.

The probe may be used in conjunction with another probe, for example a probe capable of visualising a particular tissue or a tumour. The two (or more) probes may be administered together, separately or sequentially.

The imaging probe of the present invention may be used to diagnose, assess or monitor the progression or treatment of a disease or condition.

The imaging probe of the present invention may be used to investigate the effects of a test compound on mitochondrial energisation. For example, the imaging probe may be administered together with a test compound, to and the effect of the test compound on mitochondrial energisation be assayed in real time in vivo using a method in accordance with the present invention.

### Disease

The disease or condition may be characterized by a change in mitochondrial energisation. For example, a change in mitochondrial energisation (either a higher or lower mitochondrial membrane potential) may be a symptom of the disease or may be the, or one of the, causative factors of the disease.

Full or partial reversal of the pathogenic mitochondrial energisation state following treatment may be indicative of therapeutic efficacy.

Mitochondrial oxidative damage contributes to many pathologies because mitochondria are a source of reactive oxygen species and are also susceptible to oxidative damage.

Various diseases and conditions are associated with dysfunctional mitochondria, including various cancers, diabetes, heart failure, cardiovascular and liver diseases, AIDS, degenerative diseases, immune disorders, aging and other myopathies.

The present invention provides probes that are taken up by mitochondria, the uptake being proportional to ΔΨₘ. This allows detection and imaging of dysfunctional mitochondria, for example mitochondria with suppressed or enhanced activity. Tumours commonly have a higher mitochondrial membrane potential, whereas areas of tissue damage may have a lower ΔΨₘ.

The condition and/or its treatment may be characterised by increased or decreased apoptosis, which may be monitored using an imaging probe according to the present invention. Loss of mitochondrial membrane potential is an early event in cell death caused by pro-apoptotic agents. Mitochondria-controlled apoptosis is thought to underlie cell loss in heart failure

The imaging methods of the invention may also be used to assess the efficacy of chemotherapy or radiation treatment protocols used to retard or destroy cancer and other malignant tumours.

The imaging methods of the present invention may be used to diagnose or assess cancer, for example lung, breast or prostate cancer.

It has been demonstrated that the mitochondrial transmembrane potential in carcinoma cells is significantly higher than in normal epithelial cells. For example, the difference in ΔΨm between the CX-1 colon carcinoma cell line and the control green monkey epithelial cell line CV-1 was approximately 60 mV (163 mV in tumour cells versus 104 mV in normal cells).

### Subject

The subject may be human or animal subject. The subject may be a healthy subject or a subject having or at risk from contracting a disease.

In particular the subject may have or be at risk from contracting one of the diseases or conditions mentioned in the previous section.

The subject may be undergoing treatment for the disease. The imaging probe of the invention may be used to investigate changes in mitochondrial energisation which are associated with progression of or amelioration of the disease or condition.

The subject may be an experimental animal, in particular and animal model of one of the diseases or conditions mentioned in the previous section.

### Probe Synthesis

The PET nucleus, for example ¹⁸F, may be incorporated into a precursor form of the imaging probe which is capable of receiving or adapted to receive the PET nucleus.

For example, ¹⁸F may be synthesised in a cyclotron by methods known in the art. After synthesis, the ¹⁸F is commonly in the F-form and, in view of its 110 minute half-life, needs to be rapidly incorporated into the imaging probe, purified and administered to the subject.

The present invention also provides a precursor molecule adapted to receive a PET nucleus. For example, the present invention provides a precursor molecule comprising a lipophilic cation and a hydrophobic moiety which can be reacted with an anionic form of a PET nucleus to produce an imaging probe according to the present invention.

The precursor molecule may, for example, comprise a leaving group which is susceptible to nucleophilic substitution. For example the precursor molecule may comprise a mesylate, tosylate, nosylate, triflate or iodo group, which reacts with the anionic form of the PET nucleus.

The precursor molecule may be a mesylated alkyl triphenylphosphonium compound, reactable with ¹⁸F⁻ to form ¹⁸F⁻FluoroalkylTPP. FIG. 8 shows the reaction of a mesylated undecylTPP precursor with ¹⁸F⁻ to form ¹⁸F-undecylTPP.

The present invention also provides a method for producing an imaging probe which comprises the step of reacting an anionic form of the PET nucleus with such a precursor molecule. This provides a convenient one-step procedure for production of the imaging probe.

The present invention also provides a method for producing and administering an imaging probe of the invention to a subject which comprises the following steps:
(i) synthesis of ¹⁸F⁻, for example in a cyclotron
(ii) incorporation of ¹⁸F⁻ into a precursor molecule to produce an imaging probe;
(iii) optionally purifying the imaging probe; and
(ii) administration of the optionally purified imaging probe to the subject.

The probe should be administered to the subject as soon as possible after its synthesis.

### Increase In Uptake And Clearance From Circulation

The present inventors have found that inclusion of a hydrophobic moiety into a mitochondria-targeted imaging probe increases its uptake into tissues and the extent to which it is cleared from the circulation. The uptake relative to background circulation is greater, leading to a greater tissue/circulation ratio. This greatly enhances the sensitivity of these probes for detecting and visualising changes in mitochondrial energisation in vivo.

The present invention thus provides a method for increasing the uptake of an imaging probe which comprises a triphenylphosphonium (TPP) cation which comprises the step of increasing the hydrophobicity of the imaging probe.

The rate and/or extent of uptake may be increased 5 to 50, 10 to 40, or 20 to 30 fold when compared to the uptake of the corresponding compound which lacks the hydrophobic moiety.

Uptake may be increased, in particular into certain tissues, such as kidney, muscle, heart, liver and fat.

Differences in uptake may be measured, for example between 1 hr and 5 hrs after administration.

The present invention also provides a method for increasing the tissue:circulation ratio of an imaging probe which comprises a triphenylphosphonium (TPP) cation, which method comprises the step of increasing the hydrophobicity of the imaging probe.

The tissue:circulation ratio may be obtained by comparing the concentration of the compound in the tissue (e.g. kidney, liver, muscle or heart) with the concentration of the compound in the circulation (e.g. blood).

Clearance of the imaging probe from the circulation may be increased 5 to 50, 10 to 40, or 20 to 30 fold when compared to clearance of the corresponding imaging probe which lacks the hydrophobic moiety. The tissue/circulation ratio may be at least 50-, 80-, or 100-fold greater than that of the corresponding compound which lacks the hydrophobic moiety.

An example of an imaging probe having a hydrophobic moiety is ¹⁸F-FluoroUndecylTPP, and the corresponding imaging probe lacking the hydrophobic moiety is ¹⁸F-TPP or ¹⁸F-TPMP. As the PET nucleus is unlikely to affect uptake or clearance of the probe, the PET may be compared with the corresponding molecule lacking the hydrophobic moiety and the PET nucleus (e.g. TPP or TPMP).

Hydrophobicity may therefore be increased by introducing or increasing the length of an alkyl side chain of a triphenylphosphonium lipophilic cation, such that is has at least 5, for example between 8 and 15 carbon atoms. Hydrophobicity may also be increased by adding side chains to the alkyl groups, putting aromatic groups in the chain and adding side group to the phenyl rings on the triphenylphosphonium moiety.

The invention will now be further described by way of Examples, which are meant to serve to assist one of ordinary skill in the art in carrying out the invention and are not intended in any way to limit the scope of the invention.

### EXAMPLES

### Example 1

### Investigating the Kinetics of Organ Distribution of a Mitochondria-targeted AlkylTPP Compound Following IV Injection

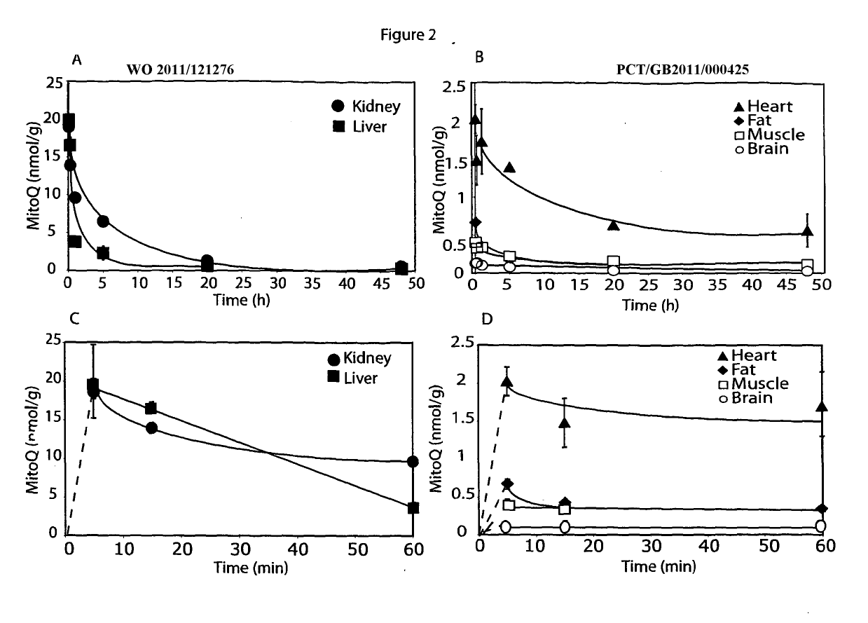

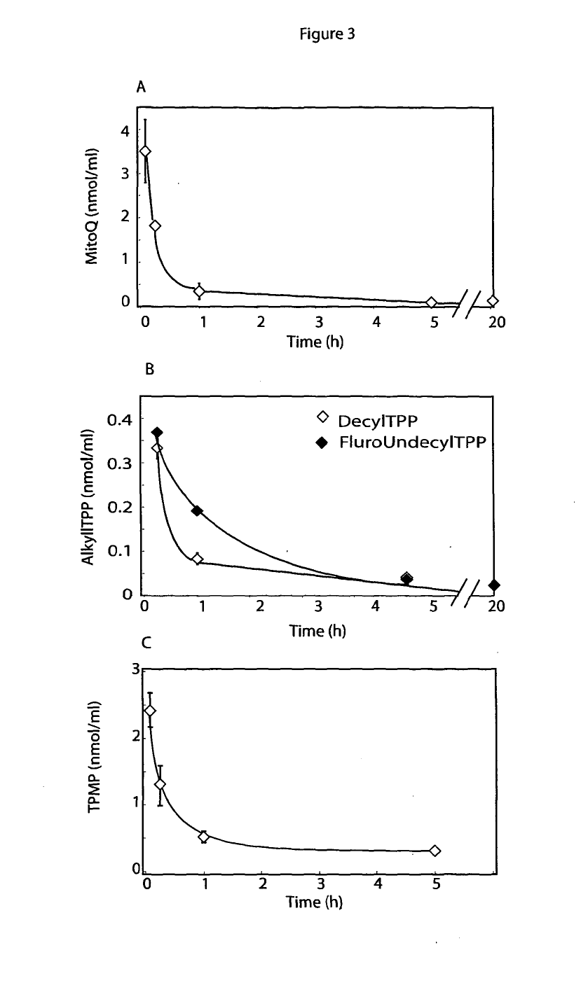

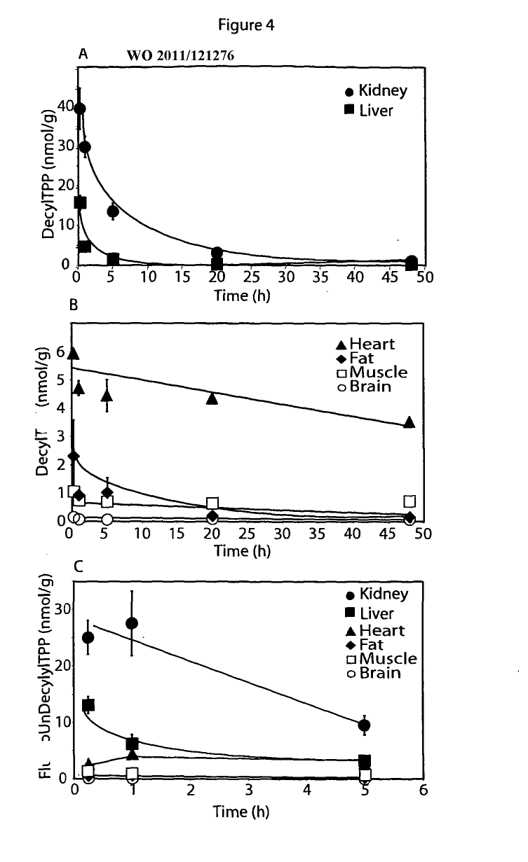

To determine how rapidly alkylTPP compounds distribute within the body, the uptake of the mitochondria-targeted antioxidant MitoQ was first assessed following intravenous (iv) injection (FIG. 2). To do this a bolus of 100 nmol [³H]MitoQ was administered to mice via tail vein injection and the amount of [³H]MitoQ in various organs was measured over the subsequent 48 h (FIG. 2). There was extensive uptake of MitoQ by the liver and kidneys when measured 1 h after injection (FIG. 2A). At this time there was less extensive uptake by the heart, lower uptake by muscle and adipose tissue and very little uptake by the brain (FIG. 2B). This rapid uptake of MitoQ into organs was mirrored by its early loss from the blood (FIG. 3A). The [³H]MitoQ accumulated by the tissues was then gradually lost with half lives of ˜2 h for the liver, ˜4 h for the kidneys and ˜15 h for the heart. There was almost complete clearance of the administered radioactivity from these tissues within 48 h, consistent with other studies where ˜80% of MitoQ was eliminated 24 h after iv injection. To assess the initial uptake of [³H]MitoQ into tissues focus was made on its tissue distribution over the first hour following injection (FIGS. 2C &

D). This showed that MitoQ uptake into tissues is very rapid, reaching its maximum at the earliest time measurable, 5 min after injection. Together these data show that MitoQ is taken up rapidly from the blood into most organs, but with significant variations in the extent of uptake by different organs.

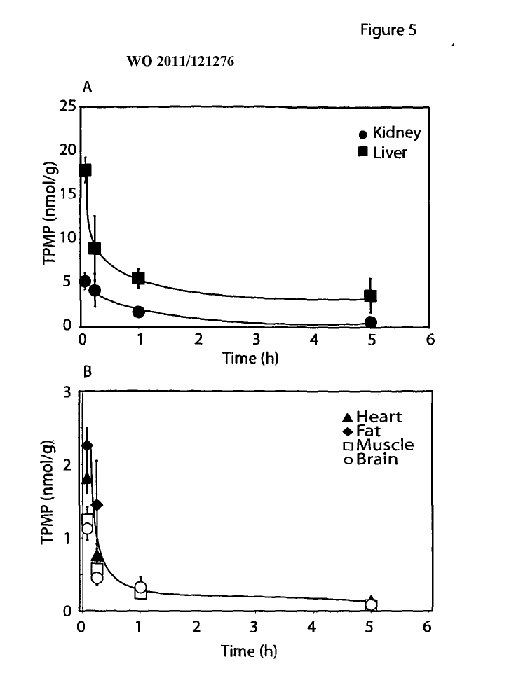

To see if the rapid uptake of MitoQ into organs from the circulation was a general property of all alkylTPP cations, or was specific to MitoQ, the organ distribution following iv injection of three alkylTPP cations, [³H]decylTPP, [³H]FluoroUndecylTPP and [³H]TPMP was assessed next. These compounds ranged in hydrophobicity from the relatively hydrophilic [³H]TPMP (Octanol/PBS partition coefficient 0.35) to the hydrophobic [³H]DecylTPP and [³H]FluoroUndecylTPP (Octanol/PBS partition coefficients of 5000 and 740±100, respectively). The tissue distribution of [³H]DecylTPP over 48 h is shown in FIGS. 4A & B, and that of [³H]FluoroUndecylTPP over 5 h is shown in FIG. 4C. Their uptake profiles were similar to that of MitoQ, with rapid uptake into the liver, kidney and heart, followed by loss with half lives of ˜3 h for both compounds from the liver and kidneys. As was the case with MitoQ, loss from the heart was slower, with half lives of ˜15 h and ˜21 h for [³H]DecylTPP and [³H]FluoroUndecylTPP, respectively. The clearance of [³H]DecylTPP and [³H]FluoroUndecylTPP from the blood (FIG. 3B) was qualitatively similar to that of [³H]MitoQ (FIG. 3A), but their blood concentrations were lower. The organ distribution of [³H]TPMP was measured for 5 h following iv injection (FIGS. 5 A,B). The [³H]TPMP distribution was qualitatively similar to those of MitoQ, DecylTPP and FluoroUndecylTPP, with rapid uptake into the liver, heart, kidneys and fat. However the extent of TPMP uptake was generally less than that of MitoQ, DecylTPP and FluoroUndecylTPP, with the exception of the brain. The half lives for loss of [³H]TPMP from the liver and kidneys were ˜3 h and ˜2 h, respectively, similar to the other TPP compounds. However, the half life for loss of TPMP from the heart was ˜2 h, significantly shorter than for the other alkylTPP compounds. The clearance of [³H]TPMP from the blood was qualitatively similar to that of the more hydrophobic alkylTPP compounds, although the concentration of [³H]TPMP in the blood remained higher (FIG. 3C). Comparing the half lives for clearance of [³H]TPMP and the other alkylTPP compounds from the remaining tissues was technically difficult due to the low amounts of compound taken up, however for both fat and muscle the half life for clearance of TPMP was estimated at ˜1-2 h, while those for DecylTPP and FluoroUndecylTPP were significantly longer at ˜7-12 h. Together these experiments demonstrated that there was extensive and rapid uptake of all alkylTPP compounds into organs from the blood.

### Example 2

### Comparison of the Extent of Uptake of AlkylTPP Compounds Into Organs Over Time

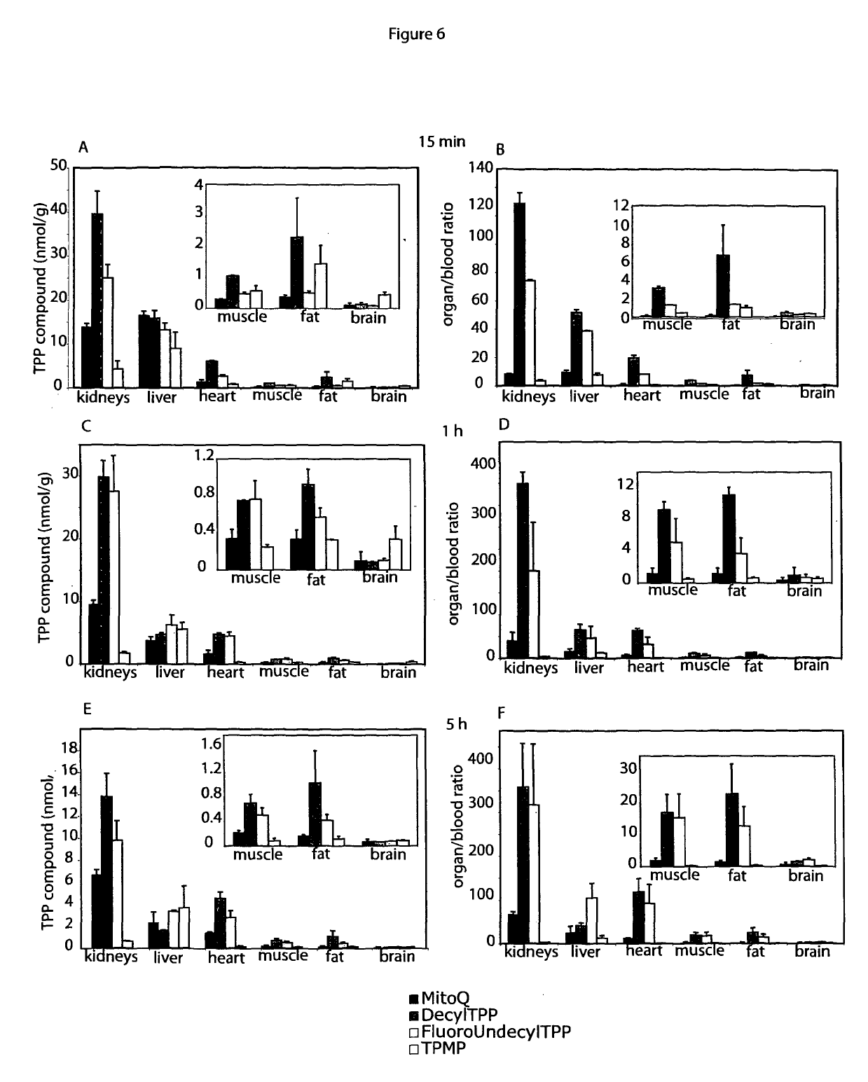

The data in FIGS. 2, 4 & 5 show that the organ distributions of the four TPP compounds assessed are qualitatively similar. However, there were significant quantitative differences between the extents of uptake of the compounds into different organs. To assess these differences we have plotted the tissue contents of the four TPP compounds at 15 min, 1 h and 5 h after iv injection (FIGS. 6 A, C, E).

As the amounts taken up into muscle, fat and brain were lower than other organs, these data are also presented as expanded inserts within each panel. This analysis showed that for the kidneys, heart and muscle the extent of uptake was in order: DecylTPP, FluoroUndecylTPP>MitoQ>TPMP, and these differences were most evident 1 to 5 h after injection. The magnitudes of these differences were large, for example the uptake of DecylTPP, MitoQ and TPMP into the heart 1 h after injection being in the ratio of 17:6:1 and by 5 h this ratio was 32:10:1. The organ distribution for MitoQ, DecylTPP and FluoroUndecylTPP were similar with uptake in the order: kidney>liver>heart>muscle, fat>brain. The organ distribution of TPMP is broadly similar to that of the three more hydrophobic compounds, with the exception that the uptake into the liver was greater than in the kidney for this compound and there was greater uptake into the brain.

AlkylTPP compounds are accumulated into tissues in response to the plasma and mitochondrial membrane potentials, as described by the Nernst equation. Consequently the concentration of the compound within mitochondria in the tissue will be determined by the mitochondrial and plasma membrane potentials, and by the concentration of compound in the circulation. As these membrane potentials are similar for all experiments reported here, a major determinant of the extent of compound uptake into tissues is its concentration in the blood. To correct for alterations in organ uptake due to this we also determined the ratio of the concentration of compound in the organ to its concentration in the circulation, and these data are shown at 15 min, 1 h and 5 h after iv injection (FIGS. 6B, D and F). These data corroborate the findings of FIGS. 6A, C and E and confirm that the extent of uptake was in order: DecylTPP, FluoroUndecylTPP>MitoQ>TPMP. For example, the heart/blood ratios for DecylTPP, MitoQ and TPMP 1 h after injection are in the ratio of 109:11:1 and by 5 h this ratio was 248:26:1. One exception is that while the liver/blood ratio of MitoQ and TPMP were similar, those for DecylTPP and FluoroUndecylTPP were significantly greater. In addition, there was little difference between the brain/blood ratio for all four compounds, suggesting that the apparent greater uptake of TPMP seen in FIGS. 6 A, C and E may arise due to its presence in entrapped blood, rather than in the brain itself.

Together, the data in FIG. 6 indicate that the organ distribution for all four compounds is qualitatively similar. DecylTPP and FluoroUndecylTPP are taken up to a significantly greater extent than MitoQ, and in turn MitoQ is taken up and retained to a far greater extent than TPMP.

### Example 3

### Uptake of AlkylTPP Compounds by Mitochondria in vivo

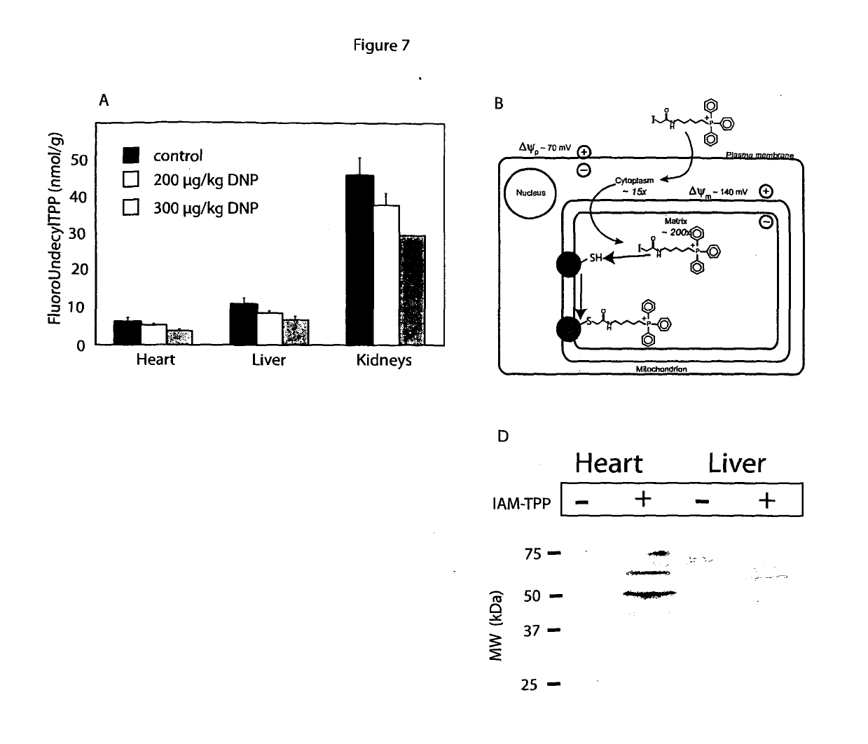

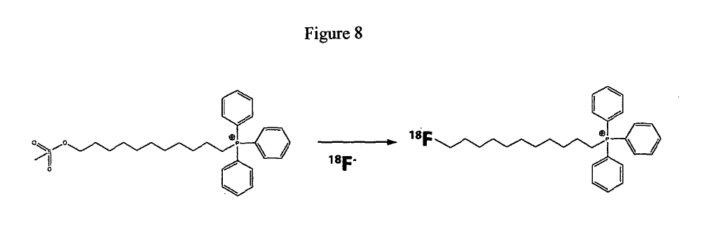

The results of Examples 1 and 2 show that alkylTPP compounds are taken up rapidly into organs in vivo. The uptake of TPP compounds into mitochondria and cells has been shown to be due to the mitochondrial and plasma membrane potentials with most of the accumulated compound being within mitochondria. Therefore it is likely that once the alkylTPP compounds are accumulated within tissues they are predominantly localised within mitochondria, driven by the membrane potential. To see if this was the case in these experiments, it was next determined if the uptake of the alkylTPP compounds in vivo was decreased by lowering the mitochondrial membrane potential. To do this, [³H]FluoroUndecylTPP was injected into mice iv and after 15 min the mice were further injected with various doses of the mitochondrial uncoupler 2,4-dinitrophenol (DNP) or saline carrier and after a further 15 min the extent of [³H]FluoroUndecylTPP accumulation by the organs was measured (FIG. 7A). The amounts of DNP used have been shown to cause partial uncoupling of mitochondria in vivo without toxicity. Our experiments showed that DNP decreased the uptake of [³H]FluoroUndecylTPP into the organs by up to 40%, in a dose dependent manner (FIG. 7A). These findings are consistent with the extent of uptake of alkylTPP compounds within organs in vivo being determined by the mitochondrial membrane potential.

To demonstrate directly the localization of alkylTPP compounds within mitochondria in vivo is technically challenging due to their rapid redistribution upon the tissue homogenisation required for mitochondrial isolation. To overcome this problem in the past, the modified alkylTPP compounds 4-iodobutylTPP (IBTP) and 10-iododecylTPP (IDTP)have been used. These compounds are accumulated by mitochondria in the same way as other alkylTPP compounds, but once within the mitochondrial matrix the iodo moiety is displaced by mitochondrial thiol proteins to form stable thioether adducts which can be visualised using antibodies against the TPP moiety. As the rates of reaction of IBTP and IDTP with protein thiols are relatively slow, this approach was extended to make a TPP compound attached to the more thiol-reactive iodoacetamide (IAM) moiety (IAM-TPP; FIG. 7B). The intention was that this molecule should be accumulated by mitochondria and there label mitochondrial thiol proteins, with the enhanced thiol reactivity of the JAM moiety facilitating the reaction of IAM-TPP with mitochondrial protein thiols in vivo before it was cleared from the organ (FIG. 7B). On incubation with isolated mitochondria IAM-TPP rapidly reacted with protein thiols and could be detected using antiTPP antibodies as was shown previously for IBTP (data not shown). Incubation of IAM-TPP with cells followed by analysis by immunocytochemistry showed uncoupler-sensitive localisation of IAM-TPP within mitochondria (FIG. 7C), confirming that IAM-TPP labelled mitochondrial proteins. A bolus of IAM-TPP was then administered to mice by iv injection and isolated mitochondria from the heart and liver 1 h later. Western blots of these mitochondrial fractions using antiserum selective for the TPP moiety showed labelling of heart and liver mitochondrial proteins by IAM-TPP (FIG. 7D). Together these data indicate that following iv injection alkylTPP compounds are rapidly taken up by mitochondria within tissues driven by the mitochondrial membrane potential.

It has thus been shown that alkylTPP compounds are accumulated by many organs in vivo within 5 min of iv administration giving therapeutically effective amounts of compound in the tissues. This uptake into tissues was due to the membrane potential-dependent accumulation of the compounds into mitochondria.

An interesting finding in this study was that DecylTPP and FluoroUndecylTPP compounds were far more extensively taken up into tissues than TPMP. This is due to the greater adsorption of the longer chain akylTPP cations to the matrix-facing surface of the mitochondrial inner membrane. Importantly, the enhancement of the extent of uptake of TPP cations by altering the side chain was large, 10 to 30-fold.

In addition to elevating the absolute levels of alkylTPP compounds within organs, modifying the alkyl side chain to a FluoroUndecyl or Decyl moiety greatly enhanced the organ/blood concentration ratio of the alkylTPP compounds by over a hundred-fold relative to TPMP. This property will be useful for the design of more effective PET probes to assess mitochondrial function in vivo in order to assess changes in mitochondrial polarisation associated with cancer and with cell death. The data presented here suggest that PET probes based on long chain, hydrophobic alkylTPP cations may have significantly better tissue loading and contrast properties than the alkylTPP cations that have been developed as PET probes to date, which are similar to TPMP. In this regard, the data obtained with FluoroUndecylTPP are particularly interesting as the ¹⁹F atom can be readily replaced by the PET-active ¹⁸F, and the uptake of FluoroUndecylTPP responded to the extent of mitochondrial polarisation in vivo.

### Example 4

### Use of 18F-UndecylTPP to Visualise Tumours and Mitochondrial Damage in Animal Models

An immunocompromised mouse model is used into which tumours of various sizes have been implanted tumours. 18F-UndecylTPP is then administered to the mice and the tumours visualised using PET.

A heart or kidney ischaemia reperfusion model is also used show that 18F-UndecylTPP may be used to visualise damaged mitochondria within tissues.

Finally, models of damage to the blood-brain barrier are used to investigate whether the probes of the invention are taken up in to the brain following this damage, thusindicating whether the blood brain barrier has been compromised.

### Materials and Methods

### Chemical Syntheses

[³H]TPMP iodide (60 Ci/mmol) was from American Radiolabeled Chemicals. [³H]MitoQ and [³H]DecylTPP preparations were synthesised and HPLC-purified to >97% radiopurity. To synthesise 11-fluoroundecyltriphenylphosphonium mesylate (FluoroUndecylTPP) a mixture of 11-bromoundecanol (752.1 mg 2.99 mmol), tetrabutylammonium fluoride (2.34 g 7.21 mmol) and H₂O (162 μL) in a Kimax tube (15 mm×150 mm), flushed with argon and sealed with a screw cap was stirred at 80° C. for 1 h. The reaction was allowed to cool slightly and extracted into pentane (25 mL). The organic layer was washed with H₂O (30 mL) three times, dried over MgSO₄, filtered and concentrated in vacuo to give 11-fluoro-1-undecanol as a slightly brown oil (416 mg 2.19 mmol 73%) containing ˜7% 11-fluoroundec-1-ene from ¹H NMR integration of the —CH₂F (4.5) vs=CH₂ (4.9-5) resonances. ¹H NMR 4.46 (2H, d,t J=47.3, 6.2 Hz, —CH₂F), 3.66 (2H, t, J=6.2 Hz, —CH₂OH), 1.2-1.8 (18H, m) ppm. ¹⁹F NMR −218.5 (t,t 24.6 Hz) ppm. A solution of crude 11-fluoro-1-undecanol (333 mg, 1.75 mmol), triethylamine (351 mg, 3.47 mmol, 484 μL, 2 equiv.) in dry CH₂Cl₂ (10 mL) was stirred at ˜10° C. for 10 min. A solution of methane sulfonyl chloride (230 mg, 2.02 mmol, 156 μL) in dry CH₂Cl₂ (1 mL) was then added while maintaining a temperature <10° C. After addition the reaction was allowed to warm to RT and stirred for 2 h. The reaction was worked up by dilution with CH₂Cl₂ (25 mL) and the organic layer was washed with H₂O (12.5 mL), 1% aqueous NaHCO₃ (12.5 mL), saturated aqueous NaCl (12.5 mL), dried over MgSO₄, filtered and concentrated in vacuo to give a slightly brown oil (444 mg). The crude product was purified by column chromatography on silica gel 60A 40-63 gm (20 g) prepared with 5% ether/pet. ether. The pure product eluted with 20% ether/pet. ether and was concentrated in vacuo to give 11-fluoroundecyl mesylate as a clear oil (279.5 mg 59%). ¹H NMR 4.43 (2H, d,t J=47.4, 6.2 Hz, —CH₂F), 4.22 (2H, t, J=6.6 Hz, —CH₂O), 3.00 (3H,s, CH3SO2), 1.6-1.8 (4H, m), 1.2-1.4 (14H, m) ppm. ¹⁹F NMR −218.4 (t,t J=47.4, 24.9 Hz) ppm. Mass Spec found: 291.1406. C₁₂H₂₅FO₃S₂Na requires: 291.1401. Purified 11-fluoroundecyl mesylate (279.5 mg, 1.041 mmol) and triphenylphosphine (582 mg, 2.21 mmol) were combined in a 10 mm×100 mm Kimax tube with a magnetic stirrer bar, flushed with Argon and sealed with a screw cap. The reaction was heated and stirred for 4 days at 90° C. The reaction products were then dissolved in minimum amount of CH₂Cl₂, concentrated in vacuo to give a crude oil (0.842 g). The crude product was redissolved in minimum CH₂Cl₂ (˜1 mL) and precipitated with ether (˜50 mL). The precipitate was allowed to settle in an ice bath and the clear liquid decanted. This dissolution/precipitation process was repeated to give 11-fluoroundecyltriphenylphosphonium mesylate as a slightly opaque, sticky oil (407.1 mg 74%). ¹H NMR: 7.69-7.81 (15H, m), 4.41 (2H, d,t J=47.4, 6.2 Hz, —CH₂F), 3.6 (2H, m, —CH₂P), 2.73 (3H, s, CH₃SO₂), 1.5-1.8 (6H, m), 1.2-1.4 (12H, m) ppm. ³¹P NMR: 24.78 ppm. ¹⁹F NMR: −218.4 (t,t J=47.4, 24.9 Hz) ppm. MS found: 435.2627, C₂₉H₃₇FP⁺ requires: 435.261.

To synthesise [³H]FluoroUndecylTPP mesylate, 11-fluoroundecyl mesylate (6.8 mg, 25.3 μmol) and [³H]triphenylphosphine (3.2 mg, 12.2 μmol, 372 μCi were combined in a 500 μL micro reaction tube with a magnetic stirrer bar, flushed with Argon and sealed with a screw cap. The reaction was heated and stirred for 4 days at 90° C. The reaction products were then dissolved in CH₂Cl₂ (40 μL) and added to diethylether (2 mL) in a capped centrifuge tube. The suspension was centrifuged at 18 160×g for 10 min and the solvents decanted. The residue was dissolved in CH₂Cl₂ (40 μL) added to ether (2 mL) and centrifuged as above. Decantation of the solvents gave the product as an oil (196 μCi). TLC on silica gel in 1:9 methanol/CH₂Cl₂ showed 99% of the radioactivity was contained between R_{f} 0.2 and 0.5.

To synthesise [5-(2-iodo-acetylamino-pentyl]triphenyl-phosohonium mesylate (IAM-TPP), a CH₂Cl₂ (10 mL) solution of 5-aminopentyltriphenylphosphonium bromide hydrogen bromide (0.050 g, 0.098 mmol) at 0° C. was added triethylamine (15 mL, 0.108 mmol) and the solution stirred for 5 minutes. The solution was cooled to −78° C. (acetone/dry ice) and solid p-nitrophenyl iodoacetate (0.030 g, 0.098 mmol) was added in one portion. The solution was stirred for 20 min and solvent removed in vacuo at −78° C. The residue was dissolved in acetone (3 mL), then sodium iodide (100 mg) was added and the solution stirred at room temperature for 2 h. The solvent was then removed in vacuo and the solid re-dissolved in CH₂Cl₂ (5 mL) and washed sequentially with distilled water and 10% aqueous sodium mesylate. The organic fraction was reduced in volume (1 mL) in vacuo and precipitated by addition to diethyl ether (20 mL). The dissolution/precipitation procedure was repeated twice to remove any residual p-nitrophenol. The final solid was dissolved in water and freeze-dried to provide a yellow voluminous solid 0.0465 g (78%). Mass Spec (high resolution +ve)—found 516.0938, C₂₅H₂₈INOP⁺ requires 516.0948. ¹HNMR (CD₃OD) 1.5-1.8 (m, 6H), 2.69 (s, 3H), 3.16 (m, 2H), 3.39-3.50 (m, 2H), 3.67 (s, 2H), 7.70-7.95 (m, 15H) ppm. ¹³C NMR (CD₃OD) −1.8, 22.7 (d, J=49 Hz), 23.1 (d, J=2 Hz), 28.5 (d, J=17 Hz), 29.2, 39.5, 40.1, 119.9 (d, J=87 Hz), 131.5 (d, J=13 Hz), 134.8 (d, J=10 Hz), 136.3 (d, J=3 Hz), 171.4 ppm. ³¹P NMR (CD₃OD) −24.8 ppm.

The published octan-1-ol partition coefficients are as follows: TPMP, 0.35; MitoQ, 2760; DecylTPP, 5000. The octan-1-ol partition coefficients for FluoroUndecylTPP, (740±100) and IAM-TPP (19±1) were determined in this study as described previously.

### Administration of Compounds to Mice

Female C57/BL6 mice (20-25 g) were maintained on a 12 h light/dark cycle with ad libitum access to standard lab chow and water. At 8-10 weeks of age the mice were placed in a restraining tube and injected iv by tail vein injection with 100 nmol [³H]compound (˜400-500 nCi) in 100 μL, sterile phosphate buffered saline (PBS) supplemented with 10% DMSO. A sample of the [³H]compound solution injected was retained to calculate the specific activity, which was subsequently used to determine the tissue content of the compound. At the indicated times after injection, the mice were killed by cervical dislocation and a blood sample of ˜100-200 μL was obtained by cardiac puncture. The organs were then removed, cleared of blood, transferred to pre-chilled Eppendorf tubes on ice, weighed and stored at −80° C. until processing. The tissues taken were: heart, kidneys, liver, brain, skeletal muscle (gastrocnemius) and white adipose tissue (subcutaneous). Injection of this amount of MitoQ and other TPP cations was previously shown to be non-toxic, as was the case here. The mice were monitored after injection to ensure no pathology or distress and all procedures were carried out under the approval of the University of Otago animal ethics committee.

Extraction of [³H] Compounds from Tissues Tissues were thawed at room temperature and transferred to 50 mL Falcon tubes. Ice-cold methanol 4° C.; 1 mL/100 mg tissue wet weight) was added to the tissue and the tissue homogenised using an Ultraturrax homogeniser (2×30 s on ice).

The homogenate was transferred in 1 mL batches to 1.5 mL Eppendorf tubes and centrifuged (10,000×g for 8 min at 4° C.). The methanol extract was decanted into a 20 ml glass scintillation vial (Wheaton) and the methanol evaporated under a stream of N₂. Further ice-cold methanol (1 mL/100 mg) was added to the tissue homogenate pellets, vortexed for 1 min, centrifuged as above and the methanol extract decanted to a fresh 20 mL glass scintillation vial and evaporated. This procedure was repeated 3 more times to give 5 extracts per tissue sample. The amount of radioactivity in the fifth extract was always negligible. Scintillant (OptiPhase HiSafe II; 10 mL) was added to each vial, ³H DPM content measured in a scintillation counter (LKB Wallac 1217 Rackbeta) using appropriate quench corrections and the total amount of radioactivity per sample calculated. The specific activity of the injected [³H] compound was then used to calculate the tissue content uptake as mol compound/g wet weight tissue. The half lives of compounds in organs were determined from the first order rate constants for loss of [³H] compound from the organs, measured as the the slope of ln[TPP compound] against time for the first 5 h after iv injection. For the liver, kidneys and heart this procedure was robust due to the large amount of radioactivity accumulated. However, the lower amounts of radioactivity accumulated by the other organs made assessment more variable, so for those organs only estimates are provided.

### Analysis of IAM-TPP Mitochondrial Uptake

To assess uptake of IAM-TPP by mitochondria in vivo, mice were injected iv by the tail vein as above with 500 nmol IAM-TPP in 100 μL sterile PBS. One hour later the mice were killed by cervical dislocation and liver and heart mitochondria prepared by homogenisation followed by differential centrifugation and the protein content determined by the bicinchoninic acid assay using bovine serum albumin as a standard. Mitochondrial proteins (40 μg) were separated by reducing SDS-polyacrylamide electrophoresis on 12.5% acrylamide gels, transferred to nitrocellulose and TPP-binding proteins detected using rabbit antiserum against the TPP moiety followed by a secondary anti-rabbit IgG conjugated to horse radish peroxidase with detection by enhanced chemiluminescence. To assess uptake of IAM-TPP into mitochondria within cells, the C2C12 mouse myoblast cell line (European Collection of Animal Cell Cultures) were grown to semi-confluence on 22 mm glass cover slips in 6 well culture dishes in DMEM (Dulbecco's modified Eagle's medium) supplemented with 10% (v/v) foetal calf serum, penicillin (100 U/ml) and streptomycin (100 μg/ml). These cells were then incubated with DMEM to lacking FCS/X% FCS for 3 h with 1 μM IAM-TPP±10 μM FCCP. For some incubations 100 nM MitoTracker Orange (Molecular Probes) was added for the last 30 min of the incubation. At the end of the incubation the cells were fixed using formaldehyde and processed for immunocytochemistry and confocal microscopy. Rabbit antiserum against the TPP moiety in conjunction with a secondary antibody of Oregon Green-conjugated to anti-rabbit IgG (Molecular Probes) were used to visualise TPP within cells. Images were acquired using a confocal microscope.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in mitochondrial biology, radiology or related fields are intended to be within the scope of the following claims.

1. An imaging probe which comprises a lipophilic cation, a hydrophobic moiety and a PET nucleus.

2. An imaging probe according to claim 1, wherein the lipophilic cation is or comprises triphenylphosphonium (TPP).

3. An imaging probe according to claim 1, wherein the hydrophobic moiety comprises an aliphatic chain comprising at least 5 carbon atoms.

4. An imaging probe according to claim 3, wherein the hydrophobic moiety comprises an aliphatic chain comprising 8 to 15 carbon atoms

5. An imaging probe according to claim 4, wherein the hydrophobic moiety comprises a linear alkyl chain.

6. An imaging probe according to claim 1, wherein the hydrophobic moiety acts as a linker between the lipophilic cation and the PET nucleus.

7. An imaging probe according to claim 1, wherein the PET nucleus is selected from the group consisting of ¹¹C, ¹⁸F, ⁷⁶Br, ¹²³I, ¹²⁴I, ¹³¹I, ¹³N, and ¹⁵O.

8. A method for analysing mitochondrial membrane potential in a subject which comprises the following steps:
(i) administering an imaging probe according to any preceding claim 1 to the subject;
(ii) visualizing the probe; and
(iii) deducing the mitochondrial membrane potential.

9. A method for visualising tumours in a subject which comprises the step of analysing mitochondrial membrane potential by a method according to claim 8.

10. A method for investigating mitochondrial damage in a subject which comprises the step of analysing mitochondrial membrane potential by a method according to claim 8.

11. A method for diagnosing and/or monitoring a pathology which involves a change in mitochondrial energisation which comprises the step of analysing mitochondrial membrane potential by a method according to claim 8.

12. A method for investigating the effect of a test compound on mitochondrial potential, which comprises the steps of administering a test compound to a subject and analysing any change in mitochondrial membrane potential by a method according to claim 8.

13. (canceled)

14. A precursor molecule comprising a lipophilic cation and a hydrophobic moiety which can be reacted with an anionic form of the PET nucleus to produce an imaging probe.

15. A precursor molecule according to claim 14, which comprises a mesylate group which reacts with the anionic form of the PET nucleus.

16. A precursor molecule according to claim 14, which is a mesylated alkyl triphenylphosphonium compound, reactable with ¹⁸F⁻to form ¹⁸F⁻FluoroalkylTPP.

17. A method for producing an imaging probe according to claim 1, which comprises the step of reacting an anionic form of the PET nucleus with a precursor molecule comprising a lipophilic cation and a hydrophobic moiety.

18. A method for producing and administering an imaging probe according to claim 1 to a subject which comprises the following steps:
(i) synthesizing ¹⁸F⁻
(ii) incorporating ¹⁸F into a precursor molecule comprising a lipophilic cation and a hydrophobic moiety to produce an imaging probe;
(ii) administering the imaging probe to the subject.

19. A method for preparing an imaging probe with increased uptake properties which comprises a triphenylphosphonium (TPP) cation which comprises the step of increasing the hydrophobicity of the imaging probe.

20. A method for preparing an imaging probe with an increased tissue:circulation ratio which comprises a triphenylphosphonium (TPP) cation, which method comprises the step of increasing the hydrophobicity of the imaging probe.

21. A method according to claim 19, wherein the hydrophobicity of the imaging probe is increased by incorporating an alkyl chain having at least 5 carbon atoms.
